(19) United States
(12) Patent Application Publication
(10) Pub. No.: US 2014/0205626 A1
(43) Pub. Date: Jul. 24, 2014

(54) ANTI-INFLAMMATORY AND IMMUNE-BOOSTING COMPOSITION CONTAINING FERMENTED GREEN COFFEE BEANS WHICH ARE FERMENTED WITH MONASCUS

(71) Applicants: COSISBIO CO., LTD., Chungcheongbuk-do (KR); COSIS CO., LTD., Cheonan-si (KR); KOREA NATIONAL UNIVERSITY OF TRANSPORTATION INDUSTRY-ACADEMIC CORPORATION FOUNDATION, Chungju-si (KR)

(72) Inventors: Byung-Choong CHOI, Seoul (KR); Dong-Goo KIM, Seongnam-si (KR); Chun-Kyu PARK, Cheonan-si (KR); Ji-Young SHIN, Cheongju-si (KR); Hoon KIM, Cheongju-si (KR); Mi-Ra LEE, Cheongju-si (KR); Hee-Yeon LEE, Cheongju-si (KR); Kwang-Won YU, Cheongju-si (KR)

(21) Appl. No.: 13/912,790

(22) Filed: Jun. 7, 2013

(30) Foreign Application Priority Data
Jan. 24, 2013 (KR) 10-2013-0008236

(51) Int. Cl. A23F 5/02; A61K 36/06; A61K 36/74

(52) U.S. Cl. CPC A23F 5/02; A61K 36/74; A61K 36/06
USPC 424/195.15; 426/61; 426/45; 424/776

(57) ABSTRACT

Disclosed herein are fermented coffee beans prepared by inoculating and culturing mycelium of fungus into coffee beans. The fermented coffee beans according to the present invention may have immune-boosting and anti-inflammatory activities.

### CROSS REFERENCE TO RELATED APPLICATION

This application claims the benefit of Korean Patent Application No. 10-2013-0008236, filed on Jan. 24, 2013, entitled “Anti-Inflammatory and Immune-Boosting Composition Containing Fermented Green Coffee Beans which are Fermented with Monascus”, which is hereby incorporated by reference in its entirety into this application.

### BACKGROUND OF THE INVENTION

1. Technical Field

The present invention relates to an anti-inflammatory and immune-boosting composition containing fermented green coffee beans which are fermented with Monascus.

2. Description of the Related Art

Coffee is one of the favorite foods most widely drunk in the world, and in accordance with the spread of coffee shops and an increase in the consumption of coffee, a coffee market has continuously grown in Korea. It is known that coffee has higher contents of antioxidant ingredients such as polyphenol, and the like, to have high scavenging activity against free radicals causing cell damage, as compared with other foods. Recently, it was also reported that roasted coffee contains lipophilic antioxidants, chlorogenic acid, and the like, having neuro-protective effects at a higher content than that of green coffee bean. When considering the fact that the coffee has been widely drunk in the world as well as in Korea, in the case of developing functional coffee having improved biological activities, it is thought that coffee is helpful in improving health and has a high commercial value.

Therefore, the present inventors studied coffee beans in order to prepare functional coffee beans having improved biological activities and discovered that fermented coffee beans prepared by inoculating and culturing mycelium of a specific fungus into the coffee beans had immune-boosting and anti-inflammatory activities, thereby completing the present invention.

### SUMMARY OF THE INVENTION

The present invention has been made in an effort to provide functional coffee beans having anti-inflammatory and immune-boosting activities.

The present invention has been also made in an effort to provide an anti-inflammatory and immune-boosting composition.

According to a preferred embodiment of the present invention, there is provided fermented coffee beans prepared by inoculating and culturing mycelium into coffee beans.

According to another preferred embodiment of the present invention, there is provided a method of preparing fermented coffee beans including: preparing coffee beans; and inoculating and culturing mycelium into the coffee beans.

According to another preferred embodiment of the present invention, there is provided an anti-inflammatory and immune-boosting composition containing fermented coffee beans prepared by inoculating and culturing mycelium into coffee beans.

According to another preferred embodiment of the present invention, there is provided a method of preparing an anti-inflammatory and immune-boosting composition including: preparing coffee beans; and inoculating and culturing mycelium into the coffee beans.

### BRIEF DESCRIPTION OF THE DRAWINGS

FIG. 1 shows a preparing process of solid-state fermented coffee beans.

FIG. 2 shows a microphage activity enhancing effect of the solid-state fermented coffee beans.

FIG. 3 shows a mitogenic activity enhancing effect of the solid-state fermented coffee beans on spleen cells.

FIG. 4 shows an effect of the solid-state fermented coffee beans on intestinal immune activity.

FIG. 5 shows a nitric oxide (NO) production inhibitory activity of the solid-state fermented coffee beans.

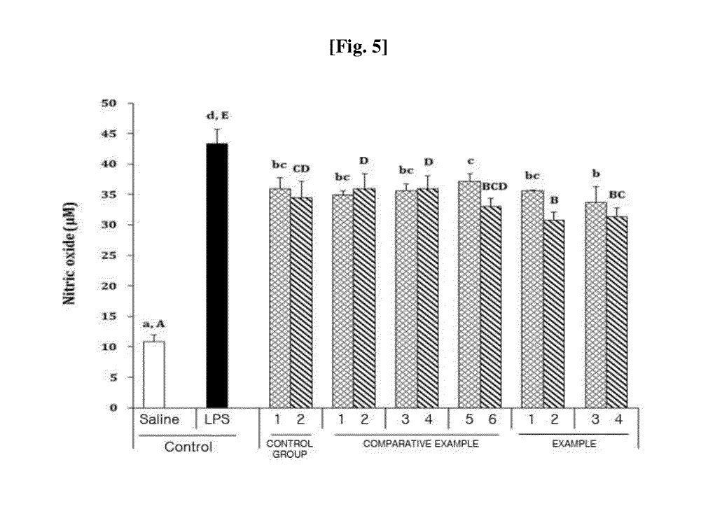

### DESCRIPTION OF THE PREFERRED EMBODIMENTS

The present invention relates to fermented coffee beans prepared by inoculating and culturing mycelium into coffee beans.

In addition, the present invention relates to a method of preparing fermented coffee beans including: preparing coffee beans; and inoculating and culturing mycelium into the coffee beans.

Further, the present invention relates to an anti-inflammatory and immune-boosting composition containing fermented coffee beans prepared by inoculating and culturing mycelium into coffee beans.

Furthermore, the present invention relates to a method of preparing an anti-inflammatory and immune-boosting composition including: preparing coffee beans; and inoculating and culturing mycelium into the coffee beans.

Hereinafter, the present invention will be described in detail.

Coffee Beans

As the coffee beans of the present invention, any coffee beans may be used as long as it is generally used, and a producing area, a variety, or a kind thereof is not particularly limited. For example, the coffee beans of the present invention may be Indonesian Mandheling coffee beans, Jamaican Blue Mountain coffee beans, Hawaiian Kona coffee beans, Costa Rican Tarrazu coffee beans, Brazilian Santos coffee beans, or the like, and appropriate coffee beans may be selected by those skilled in the art in consideration of customers' taste, cost, and the like.

The coffee beans may be preferably green coffee beans. That is, according to the present invention, the mycelium may be inoculated and cultured into the green coffee beans. When the mycelium is inoculated and cultured in a state in which the coffee beans are roasted, after the coffee beans are roasted, a time corresponding to a culturing time may be elapsed. In this case, when these coffee beans are prepared as coffee, freshness of the coffee beans and taste thereof are decreased, such that commercial values may be significantly decreased.

In addition, according to the present invention, the mycelium is inoculated and cultured into the green coffee beans instead of ground coffee beans. Since the coffee beans are prepared as coffee after the coffee beans are roasted, ground, and then powdered, the mycelium is inoculated and cultured into the ground coffee beans, which results that the mycelium is inoculated after the coffee beans are roasted. In this case, as described above, when these coffee beans are prepared as coffee, the taste is decreased, such that the commercial value may be decreased.

Mycelium

The mycelium of the present invention is mycelium of fungus. The mycelium of the present invention may be preferably mycelium of mushroom or Aspergillus, more preferably, mycelium of fungus selected from a group consisting of Monascus, Aspergillus kawachii, Aspergillus oryzae, Aspergillus niger, and most preferably, mycelium of Monascus.

Culture

According to the present invention, the mycelium are inoculated into the coffee beans and then cultured. According to the present invention, since the green coffee beans are used, solid-state culture is performed. In the case in which the coffee beans are ground and then liquid culture is performed, it is difficult to sell the fermented coffee beans after culturing as coffee beans for preparing coffee, and the fermented coffee beans becomes a fermentation product of which a main ingredient is not coffee but mycelium. In addition, after the coffee beans are roasted and ground, the ground coffee beans are processed so as to be drunk, but in the case in which the green coffee beans are roasted after the green coffee beans are firstly ground and fermented, an original taste and aroma of coffee beans may be significantly changed, and after the coffee beans are ground, oxidation such as formation of radicals, or the like, may rapidly occur. Therefore, according to the present invention, the solid-state culture needs to be performed, and the liquid-state culture should not be performed.

Fermented Coffee Beans

The fermented coffee beans according to the present invention have immune-boosting and anti-inflammatory activities. Therefore, the fermented coffee bean according to the present invention is a functional coffee bean, and in the case of preparing coffee using the fermented coffee beans, coffee having high biological activities may be obtained.

Since the fermented coffee beans according to the present invention are prepared by inoculating and culturing the mycelium into the green coffee beans, the fermented coffee beans may be sold and put into circulation, similarly to the general coffee beans. In addition, coffee may be prepared by roasting and grinding the fermented coffee beans.

Anti-Inflammatory and Immune-Boosting Composition

The anti-inflammatory and immune-boosting composition according to the present invention may contain fermented coffee beans according to the present invention obtained by inoculating the mycelium into the coffee beans.

According to the present invention, the fermented coffee beans are obtained by inoculating and solid-culturing the mycelium into the coffee beans and then solvent-extracted, such that the extracts may be used in the anti-inflammatory and immune-boosting composition according to the present invention. The anti-inflammatory and immune-boosting composition may become a food composition or cosmetic composition. The cosmetic composition according to the present invention may have an anti-inflammatory activity, such that the cosmetic composition may have an effect of alleviating skin trouble such as acne, or the like.

The foods of the present invention may be health assisting foods, health functional foods, functional foods, exercise supplementary foods, or the like, but are not limited thereto. That is, natural foods, processed foods, general food products to which the fermented coffee beans according to the present invention are added may be included therein. In addition, the foods of the present invention may be coffee itself prepared using the fermented coffee beans.

The food composition containing the fermented coffee beans according to the present invention as an active ingredient may be added as it is or used together with other foods or food compositions, and be appropriately used by a general method. A mixing amount of the active ingredient may be appropriately determined according to the purposes thereof (prevention or health maintaining purpose). In general, the fermented coffee beans according to the present invention may be added at a content of 0.01 to 70.00 weight %, preferably 0.01 to 30.00 weight %, and more preferably 0.01 to 10.00 weight %, based on the raw material content at the time of preparing food or beverages.

A kind of food is not particularly limited. The food composition containing the fermented coffee bean as the active ingredient may be used in formulations for oral administration such as tablets, hard or soft capsules, solutions, suspensions, or the like, and these formulations may be prepared using a generally acceptable carrier, for example, in the case of the formulation for oral administration, an excipient, a binder, a disintegrant, a lubricant, a solubilizer, a suspending agent, a preservative, a weighting agent, or the like.

Examples of the foods to which the fermented coffee bean may be added may include meat, sausage, bread, chocolates, candies, snacks, sweets, pizza, noodles, other noodles, gums, dairy products including ice cream, various soups, beverages, tea, drinks, alcoholic beverages, powder formulations, vitamin complexes, and the like, but are not limited thereto.

The cosmetic composition according to the present invention may be used in any formulations generally prepared in the art. That is, the cosmetic composition may be formulated as a solution, a suspension, an emulsion, a paste, a gel, a cream, a lotion, powder, soap, surfactant-containing cleansing, oil, a powder foundation, an emulsion foundation, a wax foundation, a spray, or the like, but is not limited thereto. In addition, the cosmetic composition according to the present invention may be prepared in a form of a skin softener, a nutrient emulsion, a nutrient cream, a message cream, an essence, an eye cream, a cleansing cream, a cleansing foam, cleansing water, a facial pack, a spray, or a powder.

Anti-Inflammatory and Immune-Boosting Method

The present invention relates to an anti-inflammatory and immune-boosting method including administering an anti-inflammatory and immune-boosting food composition containing fermented coffee bean obtained by inoculating and culturing mycelium into coffee bean to a subject. The administration may be preferably oral administration such as drinking coffee, or the like. The subject is not particularly limited, but may be, for example, a common person with weak or normal immunity.

Material and Method

As the coffee bean, Indonesian Mandheling green coffee bean, which is one of the green coffee beans, was purchased from Bali Bean Corp. (Gyeonggi-do, Korea) and used. Phellinus linteus (PL), Hericium erinaceum (HE), Ganoderma lucidum (GL), and two kinds of Monascus sp. that is, Monascus purpureus (MP) and Monascus rubber (MR) were obtained from RDA-Genebank Information Center in National Academy of Agricultural Science (Gyeonggi-do, Korea). Mycelia of the five kinds of fungi were cultured in potato dextrose agar (PDA, Difco, Kansas, USA) plate medium at 25 to 30° C. for 10 to 15 days, inoculated into a flask charged with potato dextrose broth (PDB, Difco), and cultured in a shaking incubator (Jeio Tech, Daejeon, Korea) for 4 to 7 days to prepare fungal spawn for solid-state fermenting green coffee bean. Then, the prepared fungal spawn was used in experiments.

Anti-Oxidant Ingredient Analysis and Anti-Oxidant Activity Measurement

A total polyphenol content in hot water extracts and ethanol extracts from coffee using the coffee bean solid-state fermented with the mycelia of three kinds of mushrooms and two kinds of Monascus species was measured using the Folin-Ciacalteu method (Velioglu Y. S., Mazza G., Cao L., and Oomah B. D., 1998. Antioxidant Activity and Total Phenolics in Selected Fruit, Vegetables, and Grain Products, J. Agric. Food Chem., 46: 4113-4117). That is, 2 mL of 2% Na2CO3 was added to 100 μL of extract sample in order to form alkaline conditions using the principle that when a Folin-Ciocalteus' reagent is reduced by polyphenolic compounds of the sample under alkaline conditions, the color is changed from blue to yellow, and then a reaction was carried out for 3 minutes. 100 μL of 50% Folin-Ciocalteus' reagent (Sigma-Aldrich, St. Louis, Mo., USA) was added thereto to carry out a reaction for 30 minutes, and then the reaction solution was measured at 750 nm, thereby confirming the total polyphenol content, which are anti-oxidant ingredient. After a calibration curve was obtained using tannic acid as a reference material, the total polyphenol content in the coffee extracts was expressed as mg tannic acid equivalents (TAE)/100 mg of the extract.

A total flavonoid content was measured using the principle that in the case of reacting flavonoid with alkali according to the method suggested by Jia, et al. (Jia Z., Tang M., Wu J., 1999. The Determination of Flavonoid Content in Mulberry and Their Scavenging Effects on Superoxide Radicals, Food Chem. 64: 555-559), flavan or flavonol glycosides becomes yellow. 100 μL of 10% aluminum nitrate and 100 μL of 1 M potassium acetate were added to 500 μL of the extract sample appropriately diluted with 80% ethanol and then left in a dark place for 40 minutes. Then, the changed absorbance was measured at 415 nm, and the result was expressed as mg quercetin equivalents (QE)/100 mg of extract, wherein quercetin was a reference material.

Meanwhile, when 1,1-diphenyl-2-picryl hydrazyl (DPPH, Sigma-Aldrich), which is a chemically stable free radical, reacts with an anti-oxidant, since the radical is scavenged while providing electrons and the color is changed, a degree at which the color of DPPH is changed by the extracts of the coffee bean solid-state fermented with the mycelium, using the method suggested by Cheung et al. (Cheung L. M., Cheung P C K., Ooi V E C., 2003. Antioxidant Activity and Total Phenolics of Edible Mushroom Extracts, Food Chem. 81: 249-25), thereby measuring the anti-oxidant activity. That is, after 50 μL of a sample was added to 0.2 mM DPPH radical solution, a reaction was carried out at room temperature for 60 minutes, and then a change in absorbance of the reaction solution was measured at 517 nm. The result was expressed as mg ascorbic acid equivalents antioxidant capacity (AEAC)/100 mg of extract, wherein ascorbic acid was a reference material.

Experimental Animal and Animal Cell Culture

As experimental animal, C3H/He, ICR, and BALE/c female mice (6 weeks old) were purchased from Samtako Com. (Gyeonggi-do, Korea) and put into a breeding tank. Then, purified water and pellet-feed for experimental animals (Samyang Co., Incheon, Korea) were freely supplied. Meanwhile, a RAW 264.7 cell line (murine macrophage cell line) used in cytotoxic and nitric oxide production inhibition experiments was obtained from Korean Cell Line Bank (Seoul, Korea) was continuously sub-cultured once every 2-3 days at 37° C. in a 5% CO₂ incubator (Vision Scientific, Gyeonggi, Korea) using DMEA medium containing 10% fetal bovine serum (FBS), 100 U/mL of penicillin, and 100 μg/mL of streptomycin supplemented with serum. In addition, lipopolysaccharide (LPS from Escherichia coli) used to induce inflammation for the anti-inflammation experiment was purchased from Sigma-Aldrich, and Ez-cytox used to measure mitogen and intestinal immune activities was purchased from Deail Labservice Co. (Seoul, Korea) to thereby be used in the experiment.

Immune Activity

Macrophage activity was measured through lysosomal phosphatase enzyme activity. 2 mL of 3% thioglycollate medium (Sigma-Aldrich) was injected into the abdominal cavity of ICR mouse. After 72 hours, the induced peritoneal macrophage was recovered and used in the experiment. Macrophage was washed with RPMI 1640 medium (10% FBS-RPMI) containing 100 U/mL of penicillin, 100 μg/mL of streptomycin, 1.25 μL/mL fungizone-amphothericin B, and 10% FBS, dispersed at 1×10⁶ cells/mL. Then, 200 μL of dispersed macrophage solution was seeded in each well of a 96-well plate (SPL Life Science, Gyeonggi, Korea) to form a macrophage monolayer (Conrad R. E., 1981, Induction and Collection of Peritoneal Exudates Macrophages. In Manual of Macrophage Methodology., Herscowitz B. H., Holden H. T., Ballanti J. A., Ghaffar A., eds. Marcel Dekker Incorporation, New York, N.Y., USA. pp. 5-11). After 2 hours, the conditioned medium was removed and non-adherent cell was washed with the RPMI 1640 medium three times. Next, macrophage was stimulated while seeding and culturing 180 μL of 10% FBS-RPMI and 20 μL of sample. After 24 hours, the conditioned medium was removed, and 150 μL 100 mM p-nitrophenylphosphate (Sigma-Aldrich) as a substrate and 50 μL of 0.1M citrate buffer were added to lysosomal phosphatase secreted by lysing cell membrane of the remaining macrophage with 25 μL of 0.1% Triton X-100 (Sigma-Aldrich), thereby carrying out the reaction. The macrophage activity of the sample was expressed as relative activity (%) with respect to a group treated with hot water extracts of non-fermented coffee bean (control group 1) by adding 0.2M borate buffer to terminate the reaction after 30 minutes of the reaction, and then measuring the absorbance at 405 nm (Suzuki I., Tanaka H., Konoshita A., Oikawa S., Osawa M., Yadomae T., 1990. Effect of Orally Administered β-glucan on Macrophage Function in Mice. Int. J. Immunopharmacol. 12: 675-684) using an ELISA reader (TECAN, Grodingen, Austria).

Meanwhile, in order to measure the mitogenic activity using spleen cells, after BALE/c mice were killed by cervical dislocation, spleens were sterilely isolated therefrom and ground, and red blood cell hemolysis was performed by 0.2% NaCl. Then, after the resultant was filtered with a metal mesh (#200) to recover splenocytes, the recovered splenocytes were washed with RPMI three times, and cell suspensions were prepared at 5×10⁶ cells/mL. 90 μL of the spleen cell suspension was seeded in each well of a 96-well plate and 10 μL of sample diluted at an appropriate concentration was added, followed by culturing in the 5% CO₂ incubator for 48 hours. The mitogenic activity of the sample was measured by the WST assay method (Ishiyama M., Tominaga H., Shiga M., Sasamoto K., Ohkura Y., Ueno K., 1996. A Combined Assay of Cell Viability and in Vitro Cytotoxicity with a Highly Water-Soluble Tetrazolium Salt, Neutral Red and Crystal Violet. Biol. Pharm. Bull. 19: 1518-1520) using an Ez-cytox solution diluted 10 times, and the result was expressed as relative activity (%) with respect to the group treated with the hot water extracts of non-fermented coffee bean (control group 1).

The intestinal immune activity through Peyer's patch was measured by a method suggested by Yu et al. (Yu K. W., Kiyohara H., Matsumoto T., Yang H. C., Yamada H., 1998. Intestinal Immune System Modulating Polysaccharides from Rhizomes of Atractylodes Lancea. Planta Med 64: 714-719). C3H/He mice were subjected to abdominal incision, Peyer's patch on the intestinal wall was isolated and ground, followed by filtering using the metal mesh (#200), thereby preparing Peyer's patch cell solutions. After the cell suspension was washed with 10% FBS-RPMI and a cell concentration was controlled at 2×10⁶ cells/mL, 180 μL of the cell suspension was seeded in each well of a 96-well plate, and 20 μL of appropriately diluted sample was added thereto, followed by culturing for 5 days. Then, only the conditioned medium was recovered and used in a bone marrow cell proliferation experiment. After the bone marrow cell was recovered from femoral bone of the same mouse, filtered, and washed and a cell concentration was controlled at 2.5×10⁵ cells/mL, 100 μL of the bone marrow cell solution was seeded in each well, and 50 μL of the conditioned medium was added thereto, followed by re-culturing for 6 days. The intestinal immune activity of the sample through Peyer's patch was expressed as relative activity (%) with respect to the group treated with the hot water extracts of non-fermented coffee beans (control group 1) by adding 10 μL of the Ez-cytox solution to the culture medium, carrying out the reaction for 6 hours, and then measuring the absorbance at 450 nm.

Nitric Oxide Production Inhibitory Activity

In order to measure the nitric oxide production inhibitor activity of the hot water extracts and ethanol extracts of coffee prepared by roasting and grinding the coffee bean solid-state fermented with mycelia of the mushrooms and Monascus, firstly, whether the sample is toxic or not was confirmed using the Ez-cytox solution (Ishiyama M., Tominaga H., Shiga M., Sasamoto K., Ohkura Y., Ueno K., 1996. A Combined Assay of Cell Viability and in Vitro Cytotoxicity with a Highly Water-Soluble Tetrazolium Salt, Neutral Red, and Crystal Violet. Biol. Pharm. Bull. 19: 1518-1520), and the result was expressed as cell viability (%) with respect to a group treated with normal saline.

Meanwhile, in order to measure the nitric oxide (NO) production inhibitory activity, RAW 264.7 cells were controlled at 1×10⁶ cells/mL in 10% FBS-DMEM, and 200 μL of the RAW 264.7 cell solution was seeded in each well of a 96-well plate, followed by culturing in the 5% CO₂ incubator, thereby attaching the cells. After 12 hours, the culture medium was entirely removed, and 160 μL of new 10% FBS-DMEM and 20 μL of sample were added together with each other. After 30 minutes, the resultant was treated with lipopolysaccharide (LPS), followed by culturing for 24 hours. Measurement of NO induced by LPS was performed by obtaining 50 μL of cell culture conditioned medium to measure NO using the Griess reaction method medium (Fox J B., 1979. Kinetics and Mechanisms of the Griess Reaction. Anal. Chem. 51: 1493-1502), and the result was expressed as an inhibition rate (%) with respect to the LPS treated group.

Statistical Treatment

Statistic analysis of the experimental results was performed by calculating a mean of the experimental results and a standard deviation (SD) using Statistical Package for the Social Science (SPSS), Ver. 12.0 (SPSS Inc., Chicago, Ill., USA), and the result was expressed as mean±SD. Analysis of Variance (ANOVA) was performed, and significance between each of the measurement values was verified by Duncan's multiple range test.

Preparation of Solid-State Fermented Coffee Beans

After the green coffee bean was immersed in water and high pressure sterilized at 121° C. for 120 minutes, spawns of mycelia of three kinds of mushrooms (Phellinus linteus, Hericium erinaceum, Ganoderma lucidum) and two kinds of Monascus sp. (Monascus purpureus and Monascus rubber) were inoculated at 10% (W/V). In addition, Phellinus linteus and Hericium erinaceum were solid-state cultured at 30° C., and Ganoderma lucidum, Monascus purpureus, and Monascus rubber were solid-state cultured at 25° C., respectively. After 4 to 12 days of the culture, the green coffee beans solid-state fermented with the fungal mycelia were dried in a drying oven (Jeio tech.) at 50° C. for 48 hours to remove moisture, and states of the mycelia inoculated into the coffee beans were observed with the naked eyes, thereby determining an optimal culture time for growth of each of the mycelia. Then, the solid-state fermented green coffee bean was prepared according to the optimal culture times of the five kinds of fungal mycelia.

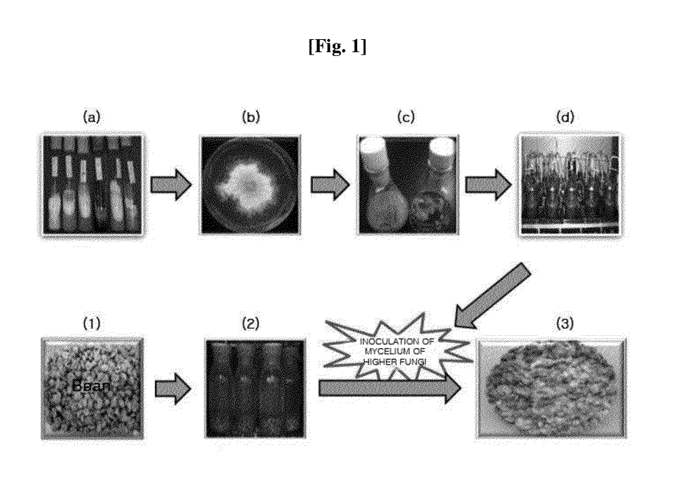

A schematic preparing process of the solid-state coffee bean was shown in FIG. 1. Various kinds of mycelia were secured (a), and the secured mycelia were activated through plate culture (b), and then optimal conditions such as a medium, a temperature, pH, and the like, were secured through a liquid-state culture (c). Thereafter, spawns of the mycelia were mass cultured using the optimal conditions (d). Meanwhile, Mandheling green coffee beans were prepared (1), and the prepared coffee beans were immersed in water and sterilized (2). Then, then the mass cultured spawns were inoculated at 10% and solid-state cultured at 25 to 30° C. for 10 days, thereby preparing the fermented coffee beans (3).

### Preparation of Examples and Comparative Examples

The green coffee beans solid-state fermented with the mycelia of three kinds of mushrooms and two kinds of Monascus sp. were medium roasted (at 235 to 240° C. for 12 to 13 minutes) in a coffee roaster (Genecafe, Gyeonggi-do, Korea) to prepare the roasted coffee, respectively. Then, the roasted coffee was ground to have the same size using a coffee grinder (Bazzatra, Gyeonggi-do, Korea).

Among solvent extracts of the coffee, hot water extracts were obtained by adding water 20 times to the roasted and ground coffee beans solid-state fermented with five kinds of mycelia, performing extraction for 2 hours using a decoction method so that a volume was halved, and removing residues using a filter (No. 2). Insoluble impurities were removed from the extraction filtrate by centrifugation (7,600×g, 4° C., 30 minutes), and the supernatant was concentrated and freeze-dried, thereby preparing the hot water extracts of the coffee beans solid-state fermented with five kinds of fungal mycelia.

In the case of ethanol extracts, ethanol (95%) was added 10 times to the roasted and ground coffee beans solid-state fermented with five kinds of mycelia, extraction was performed for 2 hours using a heating mantle (Misung, Gyeonggi-do, Korea) by a reflux extraction method (repeating 3 times), and residues were removed by filtering. The extraction filtrate was subjected to centrifugation, and then the supernatant was concentrated and freeze-dried, thereby preparing the ethanol extracts according to the kinds of mycelia.

Meanwhile, Indonesian Mandheling green coffee bean was roasted without fermentation to prepare the roasted coffee, and the roasted coffee was extracted with hot water and ethanol. The obtained extracts were used as control groups.

The hot water extracts and ethanol extracts, which were experimental groups prepared by the above-mentioned processes, were shown according to the kinds of mycelia in the following Table 1.

TABLE 1

| | | KIND OF | EXTRACTION |
|---|---|---|---|
| | | MYCELIUM | SOLVENT |
| EXAMPLE | 1 | MONASCUS PURPUREUS | HOT WATER |
| | 2 | (MP) | ETHANOL |
| | 3 | MONASCUS RUBBER | HOT WATER |
| | 4 | (MR) | ETHANOL |
| COMPARATIVE | 1 | PHELLINUS LINTEUS | HOT WATER |
| EXAMPLE | 2 | (PL) | ETHANOL |
| | 3 | HERICIUM ERINACEUM | HOT WATER |
| | 4 | (HE) | ETHANOL |
| | 5 | GANODERMA LUCIDUM | HOT WATER |
| | 6 | (GL) | ETHANOL |
| CONTROL | 1 | — | HOT WATER |
| GROUP | 2 | — | ETHANOL |

### Experimental Example 1

After the Indonesian Mandheling green coffee beans were immersed in water and high pressure sterilized, 10 weight % of the spawns of the mycelia of the mushrooms and Monascus were inoculated and solid-state cultured, thereby obtaining the solid-state fermented coffee beans. Mycelial growth in the solid-state fermented coffee beans was measured, and as a result, the mycelial growth was excellent in all of the solid-state fermented coffee beans after 10 days of the culturing.

### Experimental Example 2

### Yield

A yield of each of the experimental groups was measured. The yield was an extraction yield and calculated by the following Equation 1.

Yield (%)=(weight of a solid content in the extracts of the solid-state fermented coffee bean/weight of the green coffee bean before fermentation)×100 <Equation 1>

As a result, the yields of the control groups 1 and 2, which were the extracts of the non-fermented coffee bean, were 25.3% and 10.3%, respectively. Meanwhile, in the cases of the extracts of the fermented coffee beans, the yields of the hot water extracts of Examples were increased by 2.1 to 4.5% and the yields of the ethanol extracts of Examples were also increased by 0.3 to 2.5% than those of Comparative Examples. It may be confirmed from these results that in the cases of coffee beans solid-state fermented with the fungal mycelia, Monascus had more excellent proliferation activity than that of the mushroom (Table 2).

In addition, as a result obtained by observing the proliferation of five kinds of mycelia with the naked eyes, it may also be confirmed that in the case in which the coffee beans were fermented with the mycelia of Monascus, the proliferation efficiency was significantly higher than that in the case in which the coffee beans were fermented with the mycelia of the mushrooms.

TABLE 2

| | | YIELD (%) |
|---|---|---|
| EXAMPLE | 1 | 22.2 |
| | 2 | 12.0 |
| | 3 | 23.1 |
| | 4 | 12.2 |
| COMPARATIVE | 1 | 20.3 |
| EXAMPLE | 2 | 11.7 |
| | 3 | 20.1 |
| | 4 | 9.5 |
| | 5 | 17.7 |
| | 6 | 9.5 |
| CONTROL | 1 | 25.3 |
| GROUP | 2 | 10.3 |

### Experimental Example 3

### Anti-Oxidant Ingredient and Activity

A total polyphenol content, a total flavonoid content, and a DPPH free radical scavenging activity were measured with respect to each of the extracts of the solid-state fermented coffee beans.

As a result, in all of the Comparative Examples except for Comparative Example 3, the total polyphenol content, the total flavonoid content, and the DPPH radical scavenging activity were significantly low, as compared with the control groups 1 and 2.

On the other hand, in Examples 1 and 3, the total polyphenol content significantly increased by 1.2 times, the total flavonoid content significantly increased by 1.8 times, and the DPPH free radical scavenging activity also significantly increased by 1.2 times, as compared with the control group 1. However, in the cases of Examples 2 and 4, the total polyphenol content and the total flavonoid content were similar to those of the control group 2, and the DPPH free radical scavenging activity was slightly lower than that of the control group 2.

Therefore, it was confirmed that the anti-oxidant ingredient and activity was increased by solid-state fermenting the green coffee bean with Monascus. In addition, in the case of the hot water extract of the coffee bean fermented with Monascus, although the yields were low (yield: control group 1: 25.3%, Example 1: 22.2%, Example 3: 23.1%), the anti-oxidation activity was increased. Therefore, it may be judged that various ingredients of the green coffee bean were converted into ingredients associated with anti-oxidation during a process of fermenting the green coffee bean with the mycelia of Monascus (Table 3).

TABLE 3

| | | | | DPPH |
|---|---|---|---|---|
| | | TOTAL | TOTAL | RADICAL |
| | | POLY- | FLAVO- | SCAVENGING |
| | | PHENOLS | NOIDS | ACTIVITY |
| | | (mg*Te/ | (mg**QE/ | (mg***AEAC/ |
| | | 100 mg) | 100 mg) | 100 mg) |
| EXAM- | 1 | 1.77 ± 0.09^{d} | 0.40 ± 0.02^{d} | 18.66 ± 0.64^{d} |
| PLE | 2 | 1.41 ± 0.06^{bc} | 0.29 ± 0.03^{b} | 12.93 ± 0.63^{b} |
| | 3 | 1.79 ± 0.02^{d} | 0.40 ± 0.03^{d} | 18.24 ± 0.66^{d} |
| | 4 | 1.47 ± 0.09^{cd} | 0.27 ± 0.03^{b} | 13.22 ± 0.56^{b} |
| COMPARA- | 1 | 1.67 ± 0.03^{b} | 0.27 ± 0.02^{b} | 12.75 ± 0.42^{b} |
| TIVE | 2 | 1.34 ± 0.03^{b} | 0.25 ± 0.02^{ab} | 13.04 ± 0.81^{b} |
| EXAM- | 3 | 1.63 ± 0.04^{c} | 0.33 ± 0.02^{c} | 14.98 ± 0.82^{c} |
| PLE | 4 | 1.51 ± 0.06^{cd} | 0.28 ± 0.03^{b} | 14.77 ± 0.86^{c} |
| | 5 | 1.20 ± 0.03^{a} | 0.23 ± 0.03^{ab} | 10.08 ± 0.18^{a} |
| | 6 | 1.18 ± 0.07^{a} | 0.20 ± 0.02^{a} | 11.04 ± 0.61^{a} |
| CONTROL | 1 | 1.44 ± 0.44^{b} | 0.22 ± 0.02^{a} | 14.84 ± 0.27^{c} |
| GROUP | 2 | 1.56 ± 0.04^{d} | 0.28 ± 0.05^{b} | 16.78 ± 0.44^{d} |

*TE: tannic acid equivalents
**QE: quercetin equivalents
***AEAC: ascorbic acid equivalent antioxidant capacity

The results obtained by repeating the experiment 4 times were expressed as mean±SD, and different superscript letters mean that the standard deviations between each of the columns were significantly different (p<0.05).

### Experimental Example 4

### Immune-Boosting Activity

After the solvent extracts of the solid-state fermented coffee beans were dissolved in distilled water at a concentration of 100 μg/mg, the immune activities were measured.

As a result, the ethanol extracts had relatively low activities as compared with the hot water extracts in view of the immune activity such as the microphage activity, the mitogenic activity, the intestinal immune activity, or the like. Therefore, it was confirmed that high molecular weight materials in the hot water extracts activity rather than low molecular weight material in the ethanol extracts were mainly associated with the immune activity.

<4-1> Macrophage Proliferation Activity

In the case of the hot water extracts of the solid-state fermented coffee bean, at a sample concentration of 100 μg/mL, in the control group 1 and Comparative Examples 1, 3, and 5, the activities were similar (0.9 to 1.0 time), but the activities in Examples 1 and 3 increased by 1.18 times and 1.32 times, respectively, as compared with Comparative Example 1.

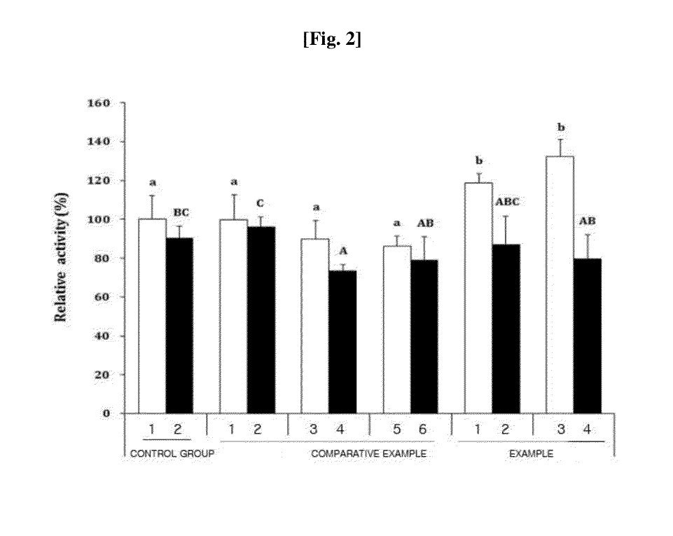

In the ethanol extracts, entirely, the activity was low as compared with the hot water extracts (the activities decreased by 0.73 to 0.90 times as compared with the control group). In the cases of the coffee beans fermented with the mycelia of Monascus, the hot water extracts had the highest activities, but in Examples 2 and 4, the activities decreased by 0.79 to 0.83 times as compared with the control group 1 (FIG. 2).

<4-2> Mitogenic Activity of Spleen Cells

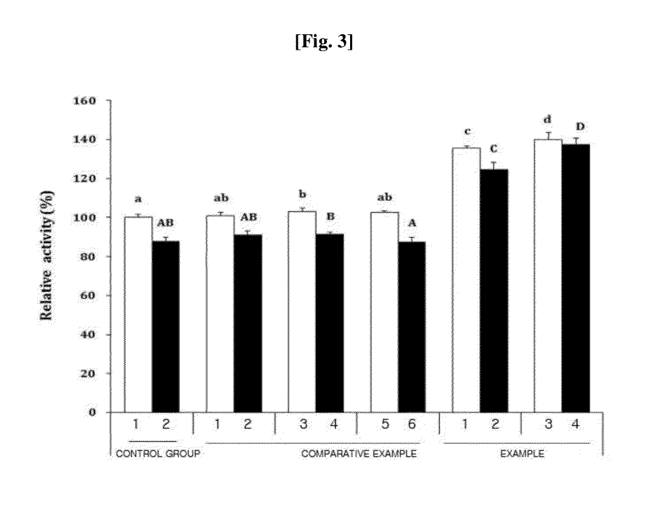

Similarly to the results of the macrophage activity experiment of <4-1>, at a sample concentration of 100 μg/mL, in Examples 1 and 3, the mitogenic activities in spleen cells significantly increased by 1.35 times and 1.40 times, respectively as compared with the control group 1. However, in Comparative Examples 1, 3, and 5, significant differences with the control group were not shown (1.00 to 1.02 times, as compared with the control group 1) (FIG. 3).

<4-3> Intestinal Immune Activity

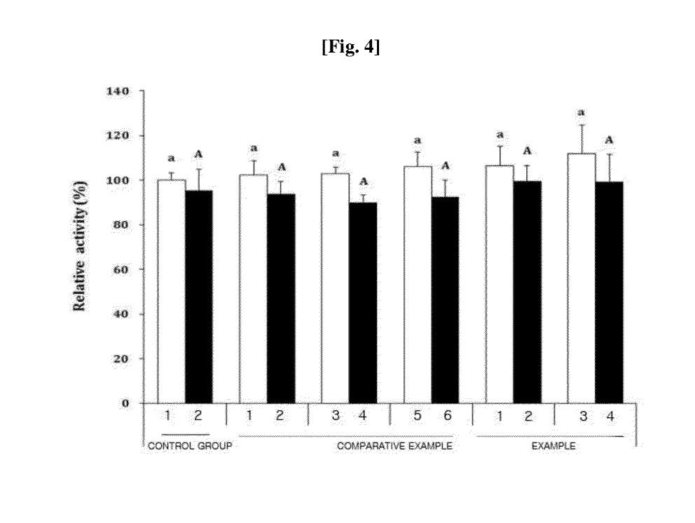

As a result of the intestinal immune activity experiment, in Example 3, the immune activity was slightly high (FIG. 4).

### Experimental Example 5

### Anti-Inflammatory Activity

Nitric oxide (NO) production inhibitory activities were measured with respect to the RAW 264.7 cell line stimulated with LPS using the solid-state fermented coffee beans as samples.

First, toxicities of the extracts to the RAW 264.7 cell line were examined, and as a result, at an extract concentration of 100 μg/mL, in the case of the ethanol extracts of the solid-state fermented coffee bean, a significant difference with the normal saline treated group was not shown, and in the case of the hot water extracts, high viability of 95% or more was obtained. However, when the extract concentration was 500 μg/mL, in the control group 1 and Comparative Example 1, viability of 95% or less was significantly obtained. Therefore, the concentrations of the hot water extracts and ethanol extracts for evaluating the NO production inhibitory activity on the RAW 264.7 cell line were controlled to 100 μg/mL, which was a concentration at which cytotoxicity was not expressed, and then the experiment was performed (Table 5).

The NO production inhibitory activity with respect to the RAW 264.7 cell line induced by LPS was calculated by the following Equation 2.

In the cases of the hot water extracts of the coffee bean, in Example 3, the NO production inhibitory activity was highest (29.9%), in the control group 1, the NO production inhibitory activity was 22.6%, and in the control group 2, the NO production inhibitory activity was 27.4%.

Meanwhile, in the cases of the ethanol extracts, in Examples 2 and 4, the NO production inhibitory activities was 38.6 and 37%, respectively, which were significantly higher than that of the control group 2.

NO production inhibitory activity=100−[{(NO content of LPS induced group−NO content of coffee bean extracts treated group)/(NO content of LPS induced group−NO content of normal saline treated group)}×100] <Equation 2>

TABLE 4

| | | *CELL VIABILITY(%) | |
|---|---|---|---|
| | | 100 μg/mL | 500 μg/mL |
| EXAMPLE | 1 | 99.86 ± 0.69^{b} | 96.20 ± 0.62^{ABC} |
| | 2 | 94.31 ± 2.42^{a} | 93.19 ± 2.47^{A} |
| | 3 | 102.77 ± 0.42^{c} | 97.32 ± 0.90^{BC} |
| | 4 | 96.63 ± 3.01^{a} | 93.58 ± 2.88^{A} |
| COMPARATIVE | 1 | 95.76 ± 1.00^{a} | 94.61 ± 2.08^{A} |
| EXAMPLE | 2 | 95.74 ± 3.42^{a} | 97.53 ± 4.98^{A} |
| | 3 | 96.92 ± 0.50^{a} | 95.22 ± 1.94^{AB} |
| | 4 | 93.28 ± 5.29^{a} | 96.61 ± 4.00^{A} |
| | 5 | 100.17 ± 0.73^{b} | 97.89 ± 0.97^{CD} |
| | 6 | 95.85 ± 3.98^{a} | 94.91 ± 3.15^{A} |
| CONTROL | 1 | 95.51 ± 1.19^{a} | 94.38 ± 1.36^{A} |
| GROUP | 2 | 93.68 ± 7.57^{a} | 98.92 ± 3.21^{A} |

| | | |
|---|---|---|
| NORMAL SALINE | 100 ± 0.87^{b,D} | 100 ± 4.42^{b,D} |
| TREATED GROUP | | |

*Cell viability (%): [(absorbance of sample)/(absorbance of normal saline treated group)] × 100

The results obtained by repeating the experiment 4 times were expressed as mean±SD, and different superscript letters (LPS treated group (100 μg/mL): small letter, LPS treated group (500 μg/mL): capital letter) mean that each of the activities were significantly different ((p<0.05).

The fermented coffee beans according to the present invention and the composition containing the same may have the high anti-inflammatory and immune-boosting activities.

What is claimed is:

1. Fermented coffee beans prepared by inoculating and culturing mycelium into coffee beans.

2. The fermented coffee beans of claim 1, wherein the mycelium is fungal mycelium.

3. The fermented coffee beans of claim 1, wherein the mycelium is mycelium of Monascus species.

4. The fermented coffee beans of claim 1, wherein the coffee beans are green coffee beans, and the culturing is solid-state culturing.

5. The fermented coffee beans of claim 1, wherein the fermented coffee beans are used to prepare coffee.

6. The fermented coffee beans of claim 1, wherein the fermented coffee beans have an anti-inflammatory or immune-boosting activity.

7. A method of preparing fermented coffee beans, the method comprising:
preparing coffee beans; and
inoculating mycelium into the coffee beans and culturing the mycelium.

8. The method of claim 7, wherein the coffee beans are green coffee beans, and the culturing is solid-state culturing.

9. The method of claim 7, wherein the fermented coffee beans have anti-inflammatory and immune boosting activities.

10. An anti-inflammatory and immune-boosting food composition comprising fermented coffee beans prepared by inoculating and culturing mycelium into coffee beans.

11. The anti-inflammatory and immune-boosting food composition of claim 10, wherein the mycelium is mycelium of Monascus.

12. The anti-inflammatory and immune-boosting food composition of claim 10, wherein the culturing is solid-state culturing.

13. The anti-inflammatory and immune-boosting food composition of claim 10, wherein the coffee beans are green coffee beans.

14. The anti-inflammatory and immune-boosting food composition of claim 10, wherein the fermented coffee beans are solvent extracts of the fermented coffee beans.
